(19) United States
(12) Patent Application Publication
(10) Pub. No.: US 2025/0322968 A1
(43) Pub. Date: Oct. 16, 2025

(54) INFORMATION PROCESSING APPARATUS, OPERATION METHOD OF INFORMATION PROCESSING APPARATUS, AND OPERATION PROGRAM OF INFORMATION PROCESSING APPARATUS

(71) Applicant: FUJIFILM CORPORATION, Tokyo (JP)

(72) Inventors: Mikinaga MORI, Kanagawa (JP); Yasushi HIKIDA, Kanagawa (JP); Mariko AKIBA, Kanagawa (JP); Issei DOI, Kanagawa (JP); Jun NAKABAYASHI, Kanagawa (JP); Kentaro NUMAJIRI, Kanagawa (JP); Makiko AIMI, Kanagawa (JP)

(21) Appl. No.: 19/098,995

(22) Filed: Apr. 3, 2025

(30) Foreign Application Priority Data
Apr. 16, 2024 (JP) 2024-065981

(51) Int. Cl. G16H 70/40

(52) U.S. Cl. CPC G16H 70/40

(57) ABSTRACT

A request reception unit receives structure information of a candidate drug by receiving a prediction request. A derivation unit derives a feature value of the candidate drug from the structure information. A prediction unit predicts an inclusion property of the candidate drug in a liposome from a contributing feature value of the candidate drug. A screen delivery controller presents a prediction result of the inclusion property of the candidate drug in the liposome and a known characteristic of a reference drug in a comparable manner.

### CROSS-REFERENCE TO RELATED APPLICATIONS

The present application claims priority under 35 U.S.C. § 119 to Japanese Patent Application No. 2024-065981, filed on Apr. 16, 2024. The above application is hereby expressly incorporated by reference, in its entirety, into the present application.

### BACKGROUND

### 1. Technical Field

The disclosed technology relates to an information processing apparatus, an operation method of an information processing apparatus, and an operation program of an information processing apparatus.

Recently, research into a drug delivery system (DDS) technique for efficiently delivering a drug to a diseased part using a pharmaceutical preparation including the drug in a vesicle such as a liposome has been actively conducted mainly for the purpose of enhancing drug efficacy and reducing side effects. For example, Ahuva Cern. et al. “Quantitative structure-property relationship modeling of remote liposome loading of drugs” Journal of Controlled Release June 2012, Volume 160 (Issue 2) p. 147-157 discloses a technique for predicting an inclusion property of a drug in a vesicle (whether or not the drug is easily included in the vesicle) using a machine learning model based on algorithms including a decision tree, a k-nearest neighbor algorithm (k-NN), and support vector regression (SVR).

### SUMMARY

In the DDS technique, it is important to know a characteristic of the drug such as the inclusion property of the drug in the vesicle or a release property of the drug from the vesicle (whether or not the drug is easily released from the vesicle) before research and development and/or manufacturing (hereinafter, collectively referred to as a practical task) of the pharmaceutical preparation. This can reduce an unnecessary cost by preventing researchers and/or manufacturers (hereinafter, collectively referred to as an operator) from trying the practical task of a pharmaceutical preparation formed of a drug that is not expected to be effective for the DDS, such as a drug having a relatively low inclusion property or a drug having a relatively high release property. The technique according to Ahuva Cern. et al. “Quantitative structure-property relationship modeling of remote liposome loading of drugs” Journal of Controlled Release June 2012, Volume 160 (Issue 2) p. 147-157 enables a prediction result of the inclusion property of the drug in the vesicle to be known before the practical task of the pharmaceutical preparation.

However, Ahuva Cern. et al. “Quantitative structure-property relationship modeling of remote liposome loading of drugs” Journal of Controlled Release June 2012, Volume 160 (Issue 2) p. 147-157 does not disclose a method of displaying the prediction result of the inclusion property of the drug in the vesicle. Thus, it is difficult for the operator to verify validity of the prediction result using the technique according to Ahuva Cern. et al. “Quantitative structure-property relationship modeling of remote liposome loading of drugs” Journal of Controlled Release June 2012, Volume 160 (Issue 2) p. 147-157. In particular, the operator involved in the practical task of the pharmaceutical preparation has knowledge about the practical task of a general preparation such as a tablet but has less knowledge about the practical task of the preparation formed of the vesicle than an expert in vesicles. This point shows that the operator may not empirically predict the characteristic of the drug related to the vesicle. Thus, it is more difficult for the operator to verify the validity of the prediction result.

One embodiment according to the disclosed technology provides an information processing apparatus, an operation method of an information processing apparatus, and an operation program of an information processing apparatus capable of contributing to verification of validity of a prediction result of a characteristic of a candidate substance included in a vesicle.

According to an aspect of the present disclosure, there is provided an information processing apparatus comprising a processor, in which the processor is configured to receive structure information of a candidate substance to be included in a vesicle, derive a feature value of the candidate substance from the structure information, predict a characteristic of the candidate substance from the feature value of the candidate substance, and present a prediction result of the characteristic of the candidate substance and a known characteristic of a reference substance in a comparable manner.

The processor is preferably configured to display, in a feature value space, a plot that corresponds to the feature value of the candidate substance and that has a display form corresponding to the prediction result.

The processor is preferably configured to display, in the feature value space, a region that corresponds to a plurality of feature values of a plurality of the reference substances and that has a display form corresponding to the known characteristic.

The processor is preferably configured to display, in the feature value space, a plurality of plots that correspond to a plurality of feature values of a plurality of the reference substances and that have a display form corresponding to the known characteristic.

The processor is preferably configured to, in accordance with reception of the structure information of a new candidate substance and derivation of a feature value of the new candidate substance, additionally display a plot corresponding to the feature value of the new candidate substance in the feature value space, in addition to the plot corresponding to the feature value of the candidate substance of which the structure information has been received so far.

The processor is preferably configured to display a plurality of plots corresponding to a plurality of the candidate substances such that the plurality of candidate substances are identifiable from each other.

The processor is preferably configured to switch the plot corresponding to the candidate substance to be displayed or not displayed in accordance with an operation instruction of an operator.

The display form is preferably a difference in color and/or pattern.

The processor is preferably configured to present coordinates of the plot corresponding to the feature value of the candidate substance in accordance with an operation instruction of an operator.

The feature value space is preferably a three-dimensional space.

The processor is preferably configured to enlarge or reduce and/or rotate the feature value space in accordance with an operation instruction of an operator.

The processor is preferably configured to present a chemical structural formula of the candidate substance in accordance with an operation instruction of an operator.

It is preferable that the feature value is composed of a plurality of types of elements, and the processor is configured to predict the characteristic of the candidate substance from a contributing feature value obtained by selecting an element contributing to prediction of the characteristic from the plurality of types of elements.

The processor is preferably configured to input the feature value of the candidate substance into a machine learning model and cause the machine learning model to output the prediction result.

It is preferable that a plurality of types of the machine learning model are prepared in accordance with a type of the vesicle, and the processor is configured to select and use the machine learning model corresponding to the type of the vesicle.

The feature value preferably includes a molecular descriptor as an element.

The characteristic preferably includes at least any one of an inclusion property of the candidate substance or the reference substance in the vesicle or a release property of the candidate substance or the reference substance from the vesicle.

The vesicle is preferably any of a liposome, a lipid nanoparticle, or a micelle.

According to another aspect of the present disclosure, there is provided an operation method of an information processing apparatus, the method comprising receiving structure information of a candidate substance to be included in a vesicle, deriving a feature value of the candidate substance from the structure information, predicting a characteristic of the candidate substance from the feature value of the candidate substance, and presenting a prediction result of the characteristic of the candidate substance and a known characteristic of a reference substance in a comparable manner.

According to still another aspect of the present disclosure, there is provided an operation program of an information processing apparatus, the program causing a computer to execute a process comprising receiving structure information of a candidate substance to be included in a vesicle, deriving a feature value of the candidate substance from the structure information, predicting a characteristic of the candidate substance from the feature value of the candidate substance, and presenting a prediction result of the characteristic of the candidate substance and a known characteristic of a reference substance in a comparable manner.

According to the disclosed technology, an information processing apparatus, an operation method of an information processing apparatus, and an operation program of an information processing apparatus capable of contributing to verification of validity of a prediction result of a characteristic of a candidate substance included in a vesicle can be provided.

### BRIEF DESCRIPTION OF THE DRAWINGS

Exemplary embodiments according to the technique of the present disclosure will be described in detail based on the following figures, wherein:

FIG. 1 is a diagram illustrating an information processing system;

FIG. 2 is a diagram illustrating a pharmaceutical preparation and a liposome;

FIG. 3 is a diagram illustrating structure information;

FIG. 4 is a block diagram illustrating computers constituting an information processing apparatus and an operator terminal;

FIG. 5 is a block diagram illustrating a processing unit of a CPU of the information processing apparatus;

FIG. 6 is a diagram illustrating a feature value;

FIG. 7 is a diagram illustrating processing of a prediction unit;

FIG. 8 is a diagram illustrating processing in a learning phase of a prediction model;

FIG. 9 is a diagram illustrating reference information;

FIG. 10 is a diagram illustrating processing of a screen delivery controller;

FIG. 11 is a diagram illustrating processing of the screen delivery controller;

FIG. 12 is a block diagram illustrating a processing unit of a CPU of the operator terminal;

FIG. 13 is a diagram illustrating a structure information input screen;

FIG. 14 is a diagram illustrating a first prediction result display screen;

FIG. 15 is a diagram illustrating a second prediction result display screen;

FIG. 16 is a diagram illustrating a state of receiving input of new structure information on the structure information input screen;

FIG. 17 is a diagram illustrating the first prediction result display screen in a case where the input of the new structure information is received;

FIG. 18 is a diagram illustrating the second prediction result display screen in a case where the input of the new structure information is received;

FIG. 19 is a diagram illustrating the second prediction result display screen on which a plot corresponding to a candidate drug of identification number 2 is not displayed;

FIG. 20 is a diagram illustrating the second prediction result display screen on which coordinates of plots are displayed;

FIG. 21 is a diagram illustrating the second prediction result display screen on which a chemical structural formula of a candidate drug of identification number 1 is displayed;

FIG. 22 is a diagram illustrating the second prediction result display screen on which a feature value space for display is enlarged;

FIG. 23 is a diagram illustrating the second prediction result display screen on which the feature value space for display is rotated;

FIG. 24 is a flowchart illustrating a processing procedure of the information processing apparatus;

FIG. 25 is a diagram illustrating an example in which a plurality of plots that correspond to a plurality of feature values of a plurality of reference drugs and that have a display form corresponding to a known characteristic are displayed in the feature value space for display;

FIG. 26 is a diagram illustrating an example in which the display form is a difference in pattern;

FIG. 27 is a diagram illustrating a plurality of types of prediction models corresponding to types of liposomes;

FIG. 28 is a diagram illustrating a state of selecting and using a prediction model corresponding to a type of the liposome;

FIG. 29 is a diagram illustrating an example of predicting a release property of a candidate drug from the liposome;

FIG. 30 is a diagram illustrating a pharmaceutical preparation and a lipid nanoparticle; and

FIG. 31 is a diagram illustrating a pharmaceutical preparation and a micelle.

### DETAILED DESCRIPTION

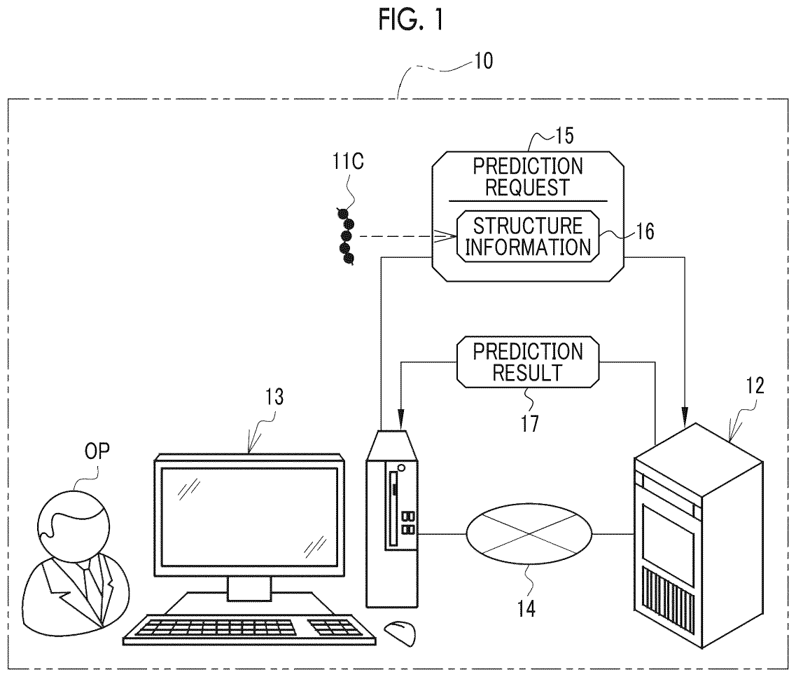

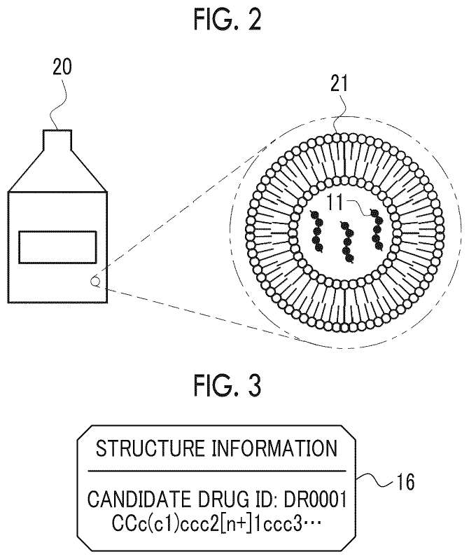

For example, as illustrated in FIG. 1, an information processing system 10 is a system that processes information related to a candidate drug 11C, and comprises an information processing apparatus 12 and an operator terminal 13. The information processing apparatus 12 and the operator terminal 13 are connected to each other through a network 14. The operator terminal 13 is installed in a pharmaceutical company that performs a practical task such as research and development and/or manufacturing of a pharmaceutical preparation 20 (refer to FIG. 2), or an organization contracted to perform the practical task of the pharmaceutical preparation 20 by the pharmaceutical company, that is, a contract research organization (CRO). The operator terminal 13 is operated by an operator OP involved in the practical task of the pharmaceutical preparation 20 in the pharmaceutical company or the contract research organization (hereinafter, collectively referred to as a pharmaceutical facility). The network 14 is, for example, a wide area network (WAN) such as the Internet or a public communication network. While only one operator terminal 13 is connected to the information processing apparatus 12 in FIG. 1, a plurality of operator terminals 13 of a plurality of pharmaceutical facilities are connected to the information processing apparatus 12 in actuality.

For example, as illustrated in FIG. 2, the pharmaceutical preparation 20 includes a liposome 21 including a drug 11. That is, the pharmaceutical preparation 20 is a so-called liposome preparation. The drug 11 is specifically an anticancer agent, an antifungal agent, an analgesic agent, or the like. The liposome 21 is a particle that is composed of at least one lipid bilayer and that has a nanoscale diameter. The candidate drug 11C is the drug 11 prepared by the operator OP as a candidate to be included in the liposome 21. Thus, the candidate drug 11C is the drug 11 before use in the practical task of the pharmaceutical preparation 20 and is the drug 11 having an unknown characteristic related to the liposome 21. The candidate drug 11C is an example of a “candidate substance” according to the disclosed technology. The liposome 21 is an example of a “vesicle” according to the disclosed technology.

With reference to FIG. 1, the operator terminal 13 transmits a prediction request 15 to the information processing apparatus 12. The prediction request 15 is a request to predict the characteristic of the candidate drug 11C via the information processing apparatus 12. The prediction request 15 includes structure information 16. The structure information 16 is information indicating a structure of the candidate drug 11C. While illustration is not provided, the prediction request 15 also includes, for example, terminal identification data (ID) for uniquely identifying the operator terminal 13 that is a transmitter of the prediction request 15.

For example, as illustrated in FIG. 3, the structure information 16 includes candidate drug ID for uniquely identifying the candidate drug 11C. The structure information 16 includes a simplified molecular input line entry system (SMILES) string of the candidate drug 11C.

With reference to FIG. 1 again, in a case where the information processing apparatus 12 receives the prediction request 15, the information processing apparatus 12 predicts the characteristic of the candidate drug 11C. A prediction result 17 of the characteristic of the candidate drug 11C is delivered to the operator terminal 13 that is the transmitter of the prediction request 15. In a case where the operator terminal 13 receives the prediction result 17, the operator terminal 13 shows the prediction result 17 to the operator OP.

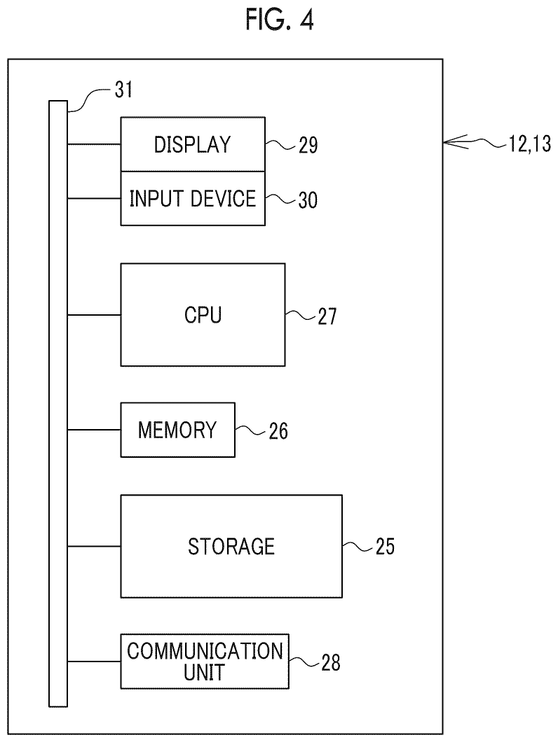

For example, as illustrated in FIG. 4, computers constituting the information processing apparatus 12 and the operator terminal 13 basically have the same configuration and each comprise a storage 25, a memory 26, a central processing unit (CPU) 27, a communication unit 28, a display 29, and an input device 30. These units are connected to each other through a busline 31.

The storage 25 is a hard disk drive that is incorporated in each of the computers constituting the information processing apparatus 12 and the operator terminal 13 or connected to the computer through a cable or a network. Alternatively, the storage 25 is a disk array obtained by connecting a plurality of hard disk drives. The storage 25 stores a control program such as an operating system, various application programs (hereinafter, referred to as an application program (AP)), various types of data associated with these programs, and the like. A solid-state drive may be used instead of the hard disk drive.

The memory 26 is a work memory for executing processing via the CPU 27. The CPU 27 loads the programs stored in the storage 25 into the memory 26 and executes processing in accordance with the programs. Accordingly, the CPU 27 controls each unit of the computer in an integrated manner. The CPU 27 is an example of a “processor” according to the disclosed technology. The memory 26 may be incorporated in the CPU 27.

The communication unit 28 is a network interface that controls transmission of various types of information through the network 14 or the like. The display 29 displays various screens. The various screens comprise an operation function based on a graphical user interface (GUI). Each of the computers constituting the information processing apparatus 12 and the operator terminal 13 receives input of an operation instruction from the input device 30 through the various screens. The input device 30 is a keyboard, a mouse, a touch panel, a microphone for audio input, or the like.

In the following description, for distinction purposes, suffix “A” will be appended to reference numerals of each unit (the storage 25 and the CPU 27) of the computer constituting the information processing apparatus 12, and suffix “B” will be appended to reference numerals of each unit (the storage 25, the CPU 27, the display 29, and the input device 30) of the computer constituting the operator terminal 13.

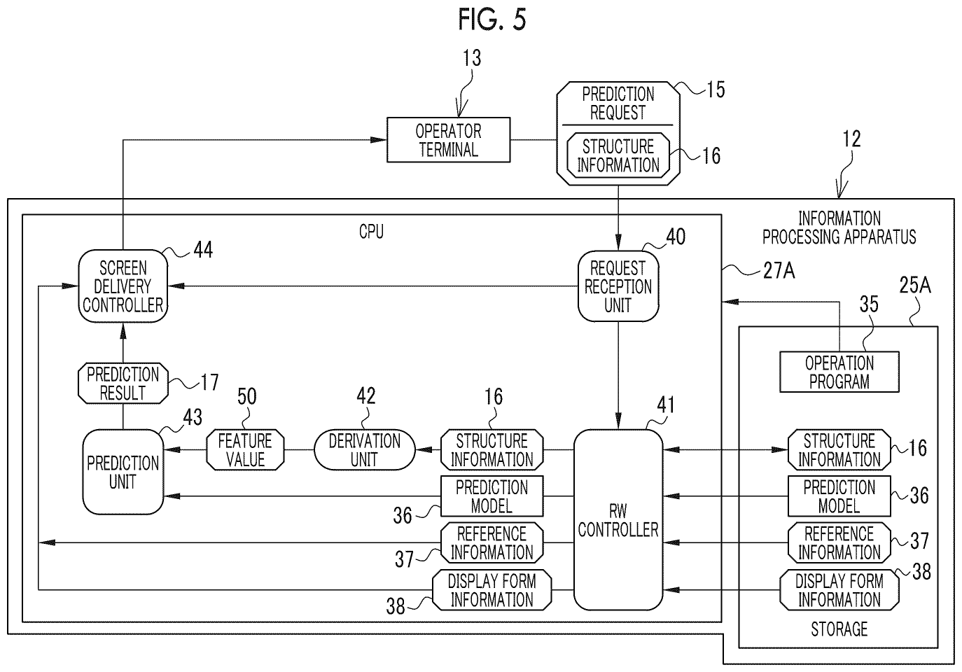

For example, as illustrated in FIG. 5, the storage 25A of the information processing apparatus 12 stores an operation program 35. The operation program 35 is an AP for causing the computer to function as the information processing apparatus 12. That is, the operation program 35 is an example of an “operation program of an information processing apparatus” according to the disclosed technology. The storage 25A also stores a prediction model 36, reference information 37, display form information 38, and the like.

In a case where the operation program 35 starts, the CPU 27A of the computer constituting the information processing apparatus 12 functions as a request reception unit 40, a read and write (hereinafter, abbreviated to RW) controller 41, a derivation unit 42, a prediction unit 43, and a screen delivery controller 44 in cooperation with the memory 26 and the like.

The request reception unit 40 receives various requests from the operator terminal 13. In particular, the request reception unit 40 receives the prediction request 15 from the operator terminal 13. As described above, the prediction request 15 includes the structure information 16. Thus, the request reception unit 40 receives the structure information 16 by receiving the prediction request 15. In a case where the request reception unit 40 receives the prediction request 15, the request reception unit 40 outputs the structure information 16 included in the prediction request 15 to the RW controller 41. The request reception unit 40 outputs the terminal ID of the operator terminal 13 included in the prediction request 15 to the screen delivery controller 44.

The RW controller 41 controls storage of various types of data in the storage 25A and readout of various types of data from the storage 25A. For example, the RW controller 41 stores the structure information 16 from the request reception unit 40 in the storage 25A. The RW controller 41 reads out the structure information 16 from the storage 25A and outputs the read structure information 16 to the derivation unit 42.

The RW controller 41 reads out the prediction model 36 from the storage 25A and outputs the read prediction model 36 to the prediction unit 43. The RW controller 41 reads out the reference information 37 and the display form information 38 from the storage 25A and outputs the read reference information 37 and the read display form information 38 to the screen delivery controller 44.

The derivation unit 42 derives a feature value 50 of the candidate drug 11C from the structure information 16. The derivation unit 42 outputs the feature value 50 to the prediction unit 43.

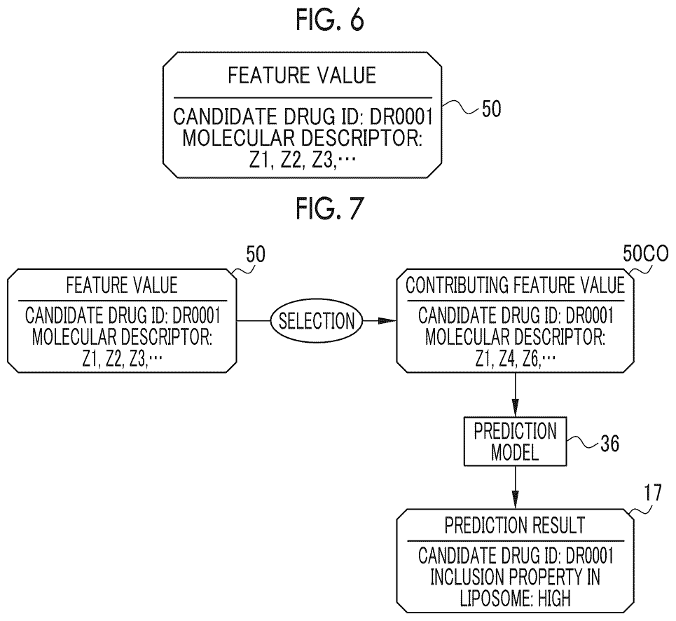

For example, as illustrated in FIG. 6, the feature value 50 includes the candidate drug ID. The feature value 50 includes a molecular descriptor as an element. The molecular descriptor is specifically a theoretical molecular descriptor. More specifically, the molecular descriptor includes a zero-dimensional descriptor such as a constitutional descriptor or a count descriptor, a one-dimensional descriptor such as a fingerprint, and a two-dimensional descriptor such as a graph invariant. The molecular descriptor includes a three-dimensional descriptor such as a weighted holistic invariant molecular (WHIM) descriptor or a quantum-chemical descriptor and a four-dimensional descriptor such as a Volsurf descriptor. That is, the feature value 50 can be said to be a multidimensional feature value vector having a plurality of types of molecular descriptors as elements. The molecular descriptor is an example of an “element” according to the disclosed technology.

With reference to FIG. 5, the prediction unit 43 outputs the prediction result 17 corresponding to the feature value 50 using the prediction model 36. The prediction unit 43 outputs the prediction result 17 to the screen delivery controller 44. The prediction model 36 is a machine learning model based on an algorithm such as support vector regression, boosting, a neural network, or a random forest. That is, the prediction model 36 is an example of a “machine learning model” according to the disclosed technology.

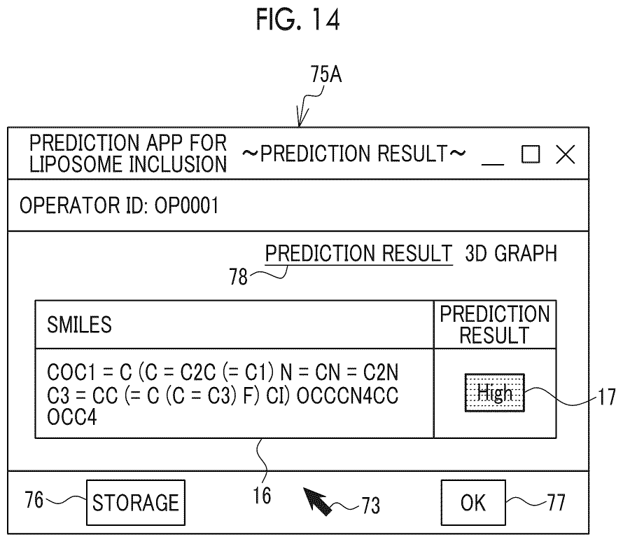

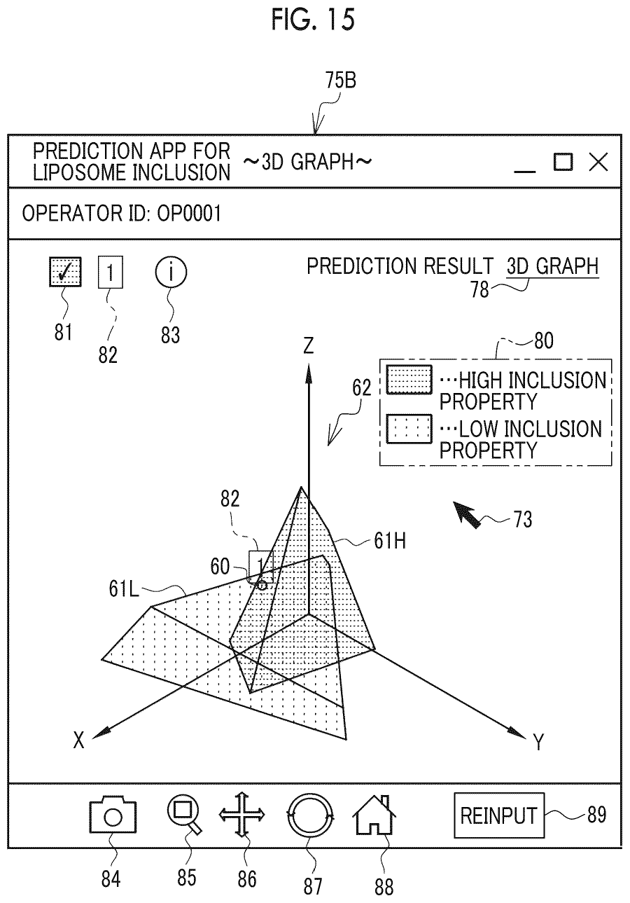

The screen delivery controller 44 performs a control of delivering the various screens to the operator terminal 13. Specifically, the screen delivery controller 44 delivers output of the various screens to the operator terminal 13 that is a transmitter of the various requests, in the form of screen data for web delivery created using a markup language such as extensible markup language (XML). In this case, the screen delivery controller 44 specifies the operator terminal 13 that is the transmitter of the various requests, based on the terminal ID from the request reception unit 40. The various screens include a structure information input screen 70 (refer to FIG. 13) for inputting the structure information 16, a first prediction result display screen 75A (refer to FIG. 14) and a second prediction result display screen 75B (refer to FIG. 15) for displaying the prediction result 17, and the like. Other data description languages such as Javascript (Registered Trademark) Object Notation (JSON) may be used instead of XML.

For example, as illustrated in FIG. 7, the prediction unit 43 selects a molecular descriptor contributing to prediction of the characteristic of the candidate drug 11C among the plurality of types of molecular descriptors constituting the feature value 50. Accordingly, the prediction unit 43 uses the feature value 50 as a contributing feature value 50CO. For example, the molecular descriptor contributing to the prediction is derived in advance as follows. That is, the prediction model 36 is caused to output the prediction result 17 by variously changing a combination of molecular descriptors to be input, and a combination of molecular descriptors having a relatively high degree of effect on the prediction result 17 is derived as the molecular descriptor contributing to the prediction.

The prediction unit 43 inputs the contributing feature value 50CO into the prediction model 36 and causes the prediction model 36 to output the prediction result 17. The prediction result 17 includes the candidate drug ID. In the present example, the characteristic of the candidate drug 11C is an inclusion property of the candidate drug 11C in the liposome 21. Thus, any of “high” or “low” for the inclusion property in the liposome 21 is registered in the prediction result 17.

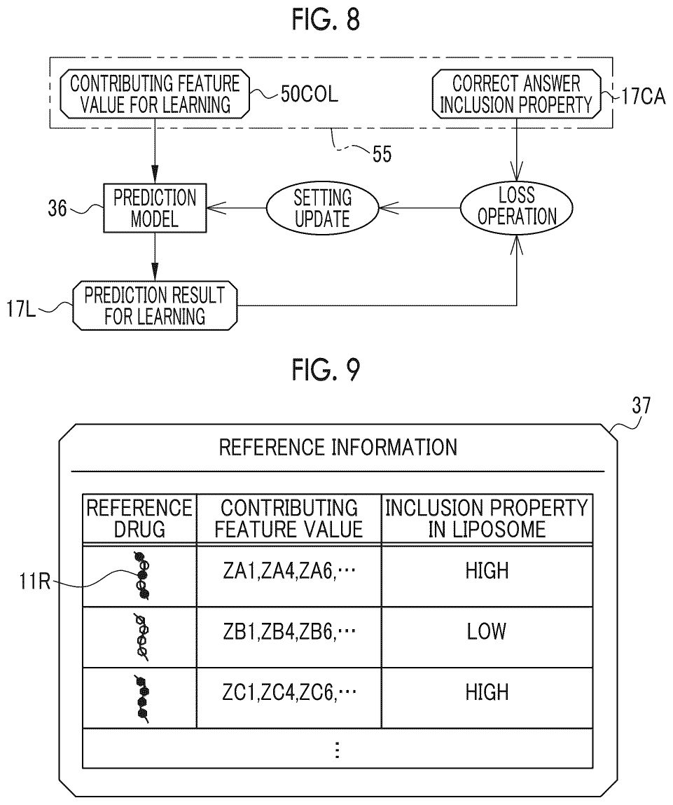

For example, as illustrated in FIG. 8, learning data (referred to as correct answer data or training data) 55 is used for training the prediction model 36. The learning data 55 is a set of a contributing feature value for learning 50COL and a correct answer inclusion property 17CA. The contributing feature value for learning 50COL is the contributing feature value 50CO of the drug 11 having a known inclusion property in the liposome 21. The correct answer inclusion property 17CA is the inclusion property, in the liposome 21, of the drug 11 that is a basis of the contributing feature value for learning 50COL. The correct answer inclusion property 17CA is so-called data for answering.

In training the prediction model 36, the contributing feature value for learning 50COL is input into the prediction model 36, and accordingly, a prediction result for learning 17L is output from the prediction model 36. The prediction result for learning 17L and the correct answer inclusion property 17CA are compared with each other, and a loss operation of the prediction model 36 using a loss function is performed based on a comparison result. Settings of various coefficients of the prediction model 36 are updated in accordance with a result of the loss operation, and the prediction model 36 is updated in accordance with the setting update.

In training the prediction model 36, the above series of processing including input of the contributing feature value for learning 50COL into the prediction model 36, output of the prediction result for learning 17L from the prediction model 36, the loss operation, the setting update, and the update of the prediction model 36 is repeated while replacing the learning data 55. Repetition of the series of processing is finished in a case where prediction accuracy of the prediction result for learning 17L reaches a predetermined set level. The prediction model 36 of which the prediction accuracy reaches the set level is stored in the storage 25A. Training may be finished in a case where the series of processing is repeated a set number of times, regardless of the prediction accuracy of the prediction result for learning 17L. The prediction model 36 may be trained in the information processing apparatus 12 or in an apparatus separated from the information processing apparatus 12. Training of the prediction model 36 may continue even after storing the prediction model 36 in the storage 25A.

For example, as illustrated in FIG. 9, the contributing feature value 50CO and the inclusion property in the liposome 21 are registered for each of a plurality of reference drugs 11R in the reference information 37. The reference drug 11R is the drug 11 that is different from the candidate drug 11C and that has a known inclusion property in the liposome 21. For example, the number of reference drugs 11R is several tens to several thousands. The inclusion property of the reference drug 11R in the liposome 21 is obtained by inputting the contributing feature value 50CO into the prediction model 36 and causing the prediction model 36 to output the prediction result 17.

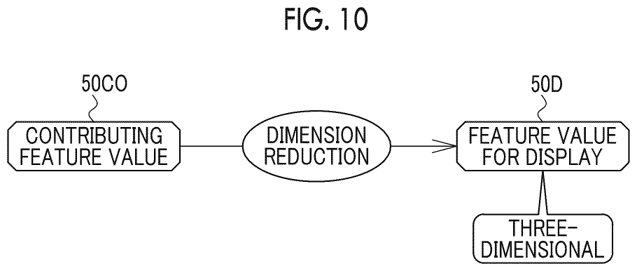

For example, as illustrated in FIG. 10, the screen delivery controller 44 reduces dimensions of the contributing feature values 50CO of the candidate drug 11C and the reference drug 11R to obtain a three-dimensional feature value for display 50D. Examples of a method of the dimension reduction include principal component analysis (PCA), t-distributed stochastic neighbor embedding (t-SNE), and uniform manifold approximation and projection (UMAP). In a case where the contributing feature value 50CO is in three or fewer dimensions, this processing of the dimension reduction is omitted.

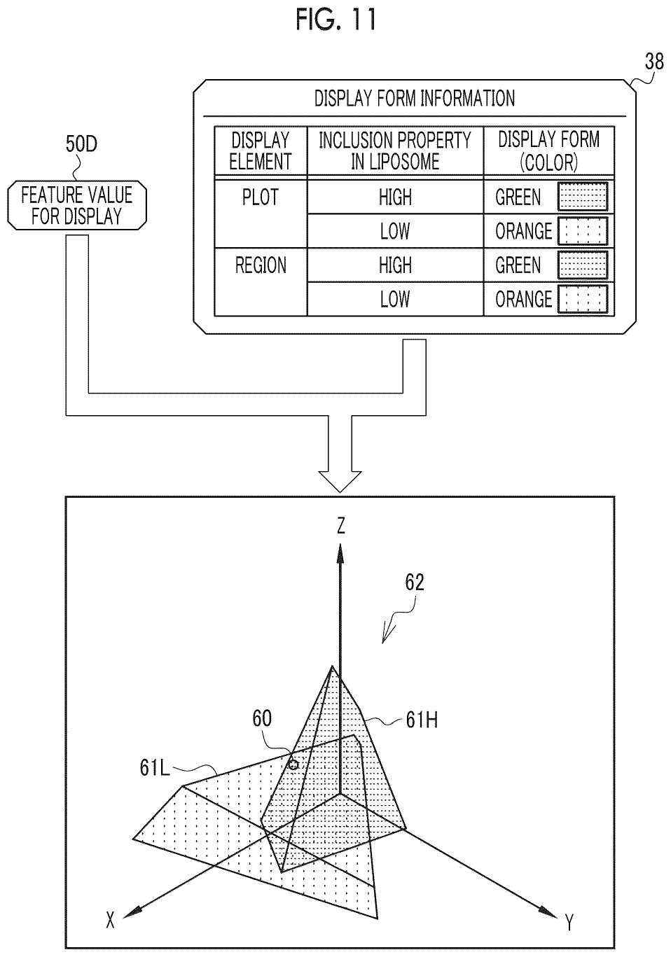

For example, as illustrated in FIG. 11, the display form information 38 defines a display form corresponding to the inclusion property in the liposome 21. In the present example, the display form is a difference in color. More specifically, a display form of a plot 60 corresponding to the feature value for display 50D of the candidate drug 11C is green in a case where the inclusion property in the liposome 21 is high, and is orange in a case where the inclusion property in the liposome 21 is low. A display form of regions corresponding to a plurality of feature values for display 50D of the plurality of reference drugs 11R is green (a region 61H) in a case where the inclusion property in the liposome 21 is high, and is orange (a region 61L) in a case where the inclusion property in the liposome 21 is low. The region 61H is a region including all of the plurality of feature values for display 50D of the reference drug 11R having a high inclusion property in the liposome 21 among the plurality of reference drugs 11R. Similarly, the region 61L is a region including all of the plurality of feature values for display 50D of the reference drug 11R having a low inclusion property in the liposome 21 among the plurality of reference drugs 11R.

The screen delivery controller 44 generates a feature value space for display 62 including the plot 60 and the regions 61H and 61L based on the feature values for display 50D of the candidate drug 11C and the reference drugs 11R and on the display form information 38. FIG. 11 illustrates a case where the inclusion property of the candidate drug 11C in the liposome 21 is high. The feature value space for display 62 is an example of a “feature value space” according to the disclosed technology.

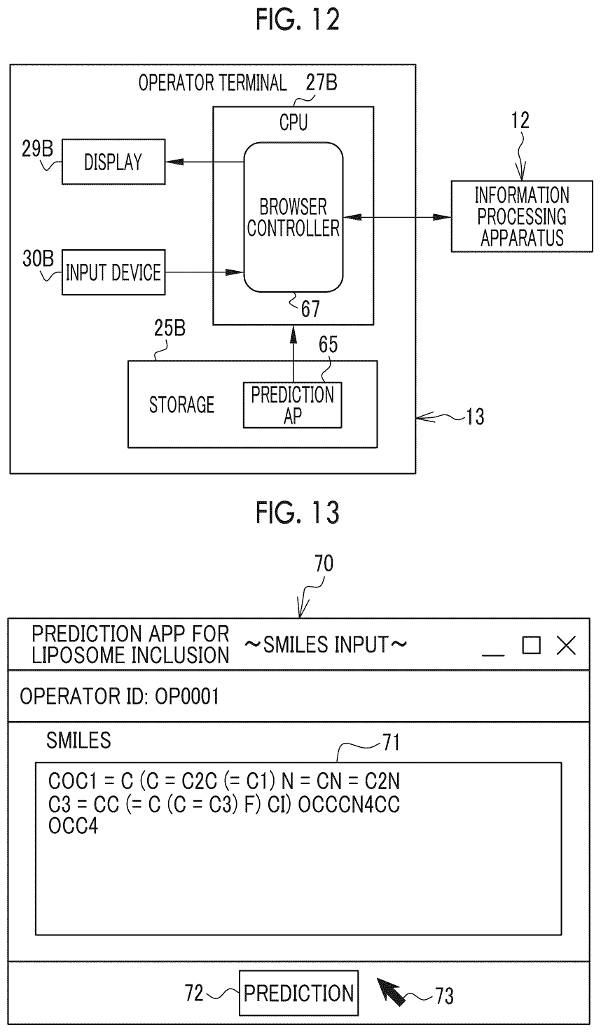

For example, as illustrated in FIG. 12, the storage 25B of the operator terminal 13 stores a prediction AP 65. The prediction AP 65 is installed on the operator terminal 13 by the operator OP. The prediction AP 65 is an AP for a service of predicting the characteristic of the candidate drug 11C via the information processing apparatus 12. In a case where the prediction AP 65 starts, the CPU 27B of the operator terminal 13 functions as a browser controller 67 in cooperation with the memory 26 and the like. The browser controller 67 controls an operation of a dedicated web browser of the prediction AP 65.

The browser controller 67 reproduces various screens based on various types of screen data from the information processing apparatus 12 and displays the reproduced various screens on the display 29B. The browser controller 67 receives various operation instructions input from the input device 30B by the operator OP, through the various screens. The browser controller 67 transmits various requests corresponding to the operation instructions including the prediction request 15 to the information processing apparatus 12.

In a case where the prediction AP 65 starts and login is performed, the structure information input screen 70 illustrated in, for example, FIG. 13 is displayed on the display 29B under control of the browser controller 67. The structure information input screen 70 is provided with an input box 71 for the structure information 16. In the input box 71, a SMILES string as the structure information 16 can be input, or a file of the SMILES string can be dropped.

The operator OP inputs the desired structure information 16 in the input box 71 and then selects a prediction button 72 using a cursor 73. In a case where the prediction button 72 is selected, the browser controller 67 generates the prediction request 15 including the structure information 16 input in the input box 71 and transmits the generated prediction request 15 to the information processing apparatus 12.

In a case where the inclusion property of the candidate drug 11C in the liposome 21 is predicted in the information processing apparatus 12, the first prediction result display screen 75A illustrated in, for example, FIG. 14 is displayed on the display 29B under control of the browser controller 67. The SMILES string, which is the structure information 16 of the candidate drug 11C, and the prediction result 17 are displayed on the first prediction result display screen 75A. FIG. 14 illustrates a case where the inclusion property of the candidate drug 11C in the liposome 21 is high and a text “High” is shown in a block indicating the prediction result 17. The block indicating the prediction result 17 has a display form corresponding to the prediction result 17, like the plot 60 and the like. In the example of FIG. 14, the block is green.

A storage button 76 and an OK button 77 are provided in a lower portion of the first prediction result display screen 75A. In a case where the storage button 76 is selected using the cursor 73, the structure information 16 and the prediction result 17 are associated with each other using the candidate drug ID and stored in the storage 25B in the form of, for example, a comma-separated values (CSV) file. In a case where the OK button 77 is selected using the cursor 73, display of the first prediction result display screen 75A is cleared.

A display switching tab 78 is provided in an upper portion of the first prediction result display screen 75A. In a case where “3D graph” of the display switching tab 78 is selected using the cursor 73, the second prediction result display screen 75B illustrated in, for example, FIG. 15 is displayed on the display 29B under control of the browser controller 67. The feature value space for display 62 including the plot 60 and the regions 61H and 61L is displayed with a legend 80 on the second prediction result display screen 75B. By displaying the feature value space for display 62 including the plot 60 and the regions 61H and 61L, the screen delivery controller 44 presents the prediction result 17 of the inclusion property of the candidate drug 11C in the liposome 21 and the known characteristic of the reference drug 11R in a comparable manner.

A checkbox 81, an identification number 82, and an information display button 83 are provided in an upper portion of the second prediction result display screen 75B. The checkbox 81 has a display form corresponding to the prediction result 17, like the plot 60 and the like. The identification number 82 is also attached to the plot 60 of the feature value space for display 62. A capture button 84, an enlarging and reducing button 85, a translation button 86, a rotation button 87, and a home button 88 are provided in a lower portion of the second prediction result display screen 75B. These GUI functions will be described later. In a case where “prediction result” of the display switching tab 78 is selected using the cursor 73 on the second prediction result display screen 75B, display is restored to the first prediction result display screen 75A.

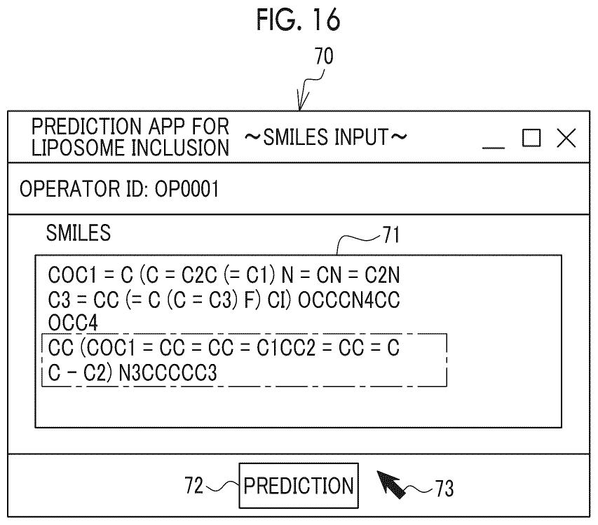

A reinput button 89 is further provided in the lower portion of the second prediction result display screen 75B. In a case where the reinput button 89 is selected using the cursor 73, display is restored to the structure information input screen 70 as illustrated in, for example, FIG. 16, and the structure information 16 of a new candidate drug 11C can be input. FIG. 16 illustrates a state where the structure information 16 of the new candidate drug 11C surrounded by a dot-dashed line is input in the input box 71 in addition to the structure information 16 illustrated in FIG. 13. The reinput button 89 may also be provided on the first prediction result display screen 75A.

In a case where the prediction button 72 is selected using the cursor 73 in the state illustrated in FIG. 16, the browser controller 67 generates the prediction request 15 including the structure information 16 of the new candidate drug 11C input in the input box 71 and transmits the generated prediction request 15 to the information processing apparatus 12.

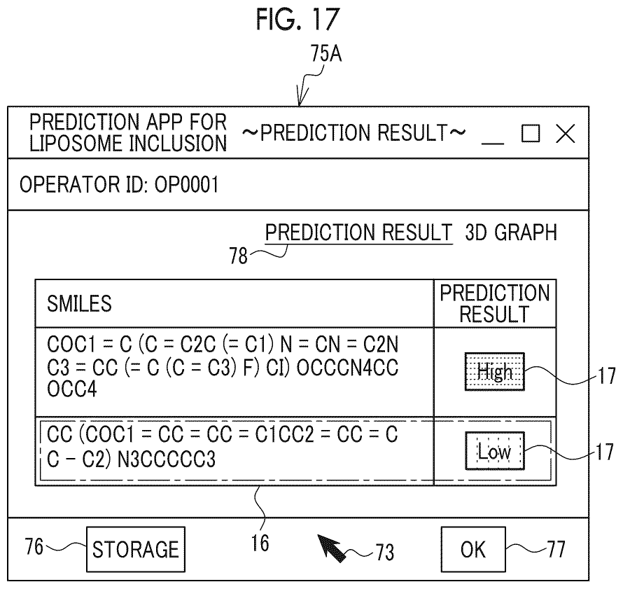

In a case where the inclusion property of the new candidate drug 11C in the liposome 21 is predicted in the information processing apparatus 12, the structure information 16 and the prediction result 17 of the new candidate drug 11C as surrounded by a dot-dashed line are additionally displayed on the first prediction result display screen 75A as illustrated in, for example, FIG. 17, in addition to the structure information 16 and the prediction result 17 of the candidate drug 11C of which the structure information 16 has been received so far.

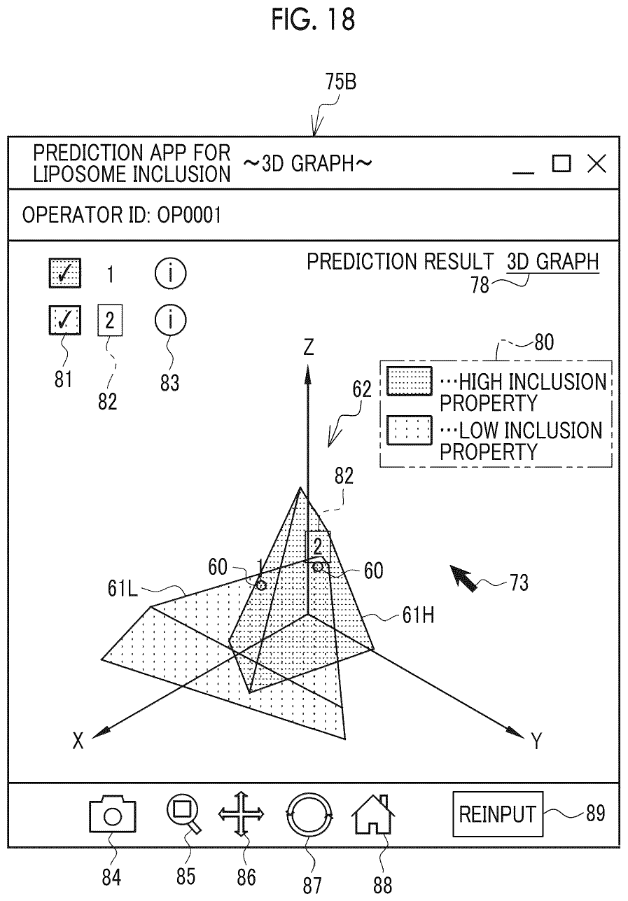

For example, as illustrated in FIG. 18, the plot 60 with the identification number 82 of “2” corresponding to the feature value for display 50D of the new candidate drug 11C is additionally displayed in the feature value space for display 62 of the second prediction result display screen 75B, in addition to the plot 60 with the identification number 82 of “1” corresponding to the feature value for display 50D of the candidate drug 11C of which the structure information 16 has been received so far. As is understood from this description, by using the identification number 82, a plurality of plots 60 corresponding to the plurality of candidate drugs 11C can be displayed such that the plurality of candidate drugs 11C can be identified from each other.

While FIGS. 16 to 18 illustrate an aspect of predicting the inclusion property of the new candidate drug 11C in the liposome 21 after predicting the inclusion property of the certain candidate drug 11C in the liposome 21, an aspect of predicting the inclusion properties of the plurality of candidate drugs 11C in the liposome 21 is not limited to this. The input of the structure information 16 of the plurality of candidate drugs 11C may be received from the beginning on the structure information input screen 70, and the inclusion property of the plurality of candidate drugs 11C in the liposome 21 may be collectively predicted. In this case, a CSV file in which a plurality of SMILES strings are registered may be received in the input box 71 as the structure information 16. Considering fairness between operators OP, a processing load of the CPU 27A, and the like, the number of candidate drugs 11C that can be collectively predicted is preferably limited by, for example, setting the number of candidate drugs 11C that can be collectively predicted to 10.

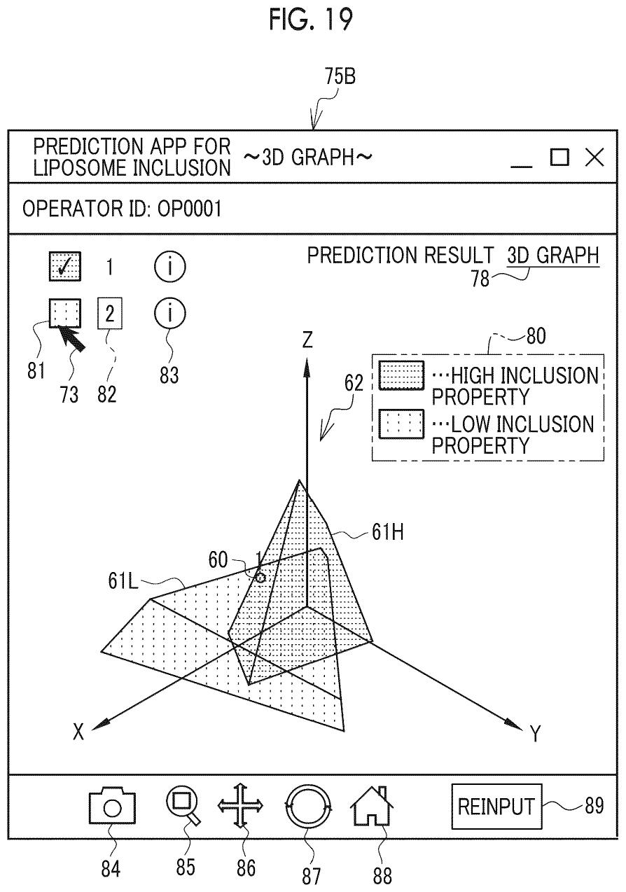

For example, as illustrated in FIG. 19, in a case where the checkbox 81 is selected using the cursor 73 to be unchecked, the plot 60 corresponding to the checkbox 81 is not displayed in the feature value space for display 62. In a case where the checkbox 81 is selected again using the cursor 73 to be checked again, the plot 60 corresponding to the checkbox 81 is displayed again in the feature value space for display 62. That is, the screen delivery controller 44 switches the plot 60 to be displayed or not displayed in accordance with the operation instruction of the operator OP for selecting the checkbox 81. FIG. 19 illustrates a case where the plot 60 with the identification number 82 of “2” is not displayed.

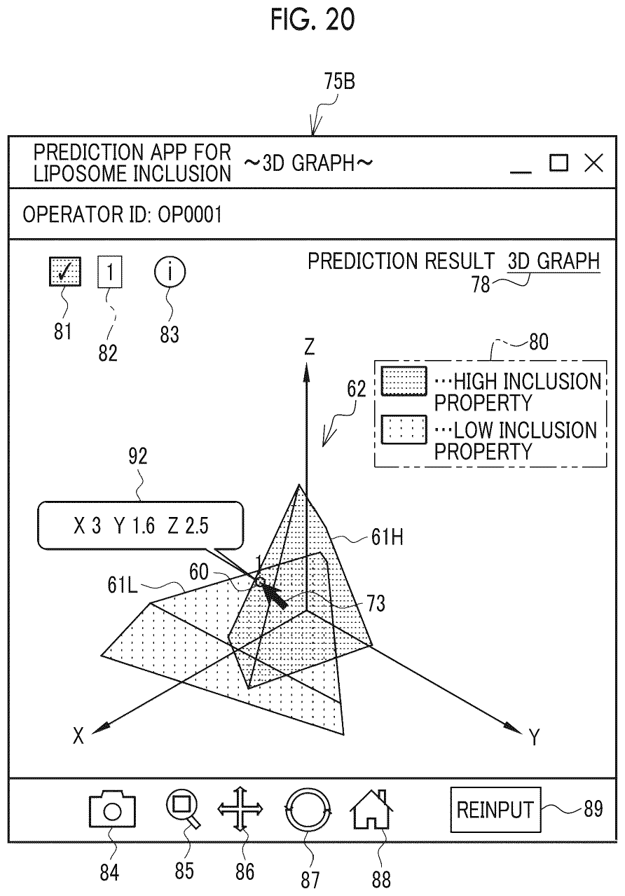

For example, as illustrated in FIG. 20, in a case where the plot 60 is selected using the cursor 73, coordinates 92 of the plot 60 in the feature value space for display 62 are displayed. That is, the screen delivery controller 44 presents the coordinates 92 of the plot 60 in accordance with the operation instruction of the operator OP for selecting the plot 60.

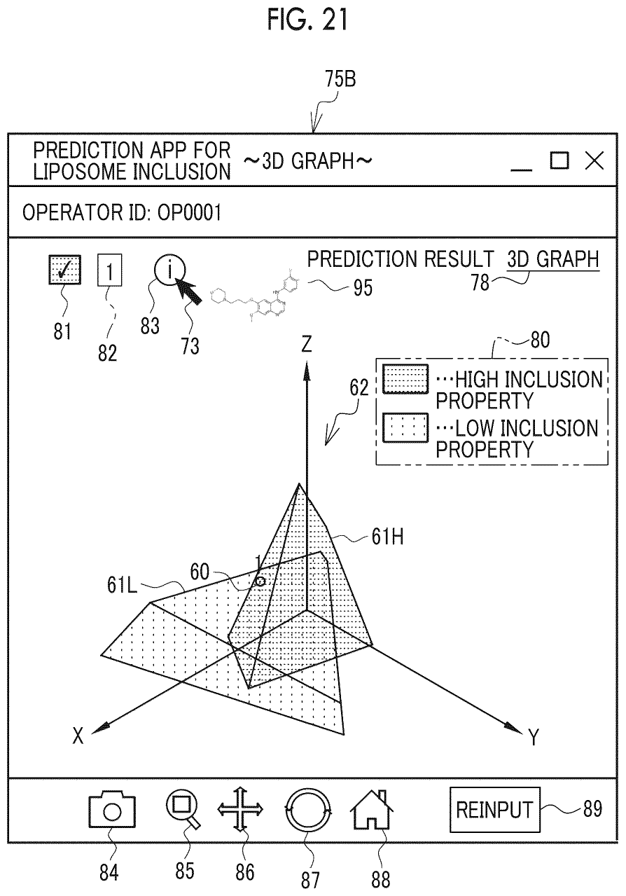

For example, as illustrated in FIG. 21, in a case where the information display button 83 is selected using the cursor 73, a chemical structural formula 95 of the candidate drug 11C corresponding to the information display button 83 is displayed. That is, the screen delivery controller 44 presents the chemical structural formula 95 of the candidate drug 11C in accordance with the operation instruction of the operator OP for selecting the information display button 83. The chemical structural formula 95 is generated from the SMILES string as the structure information 16.

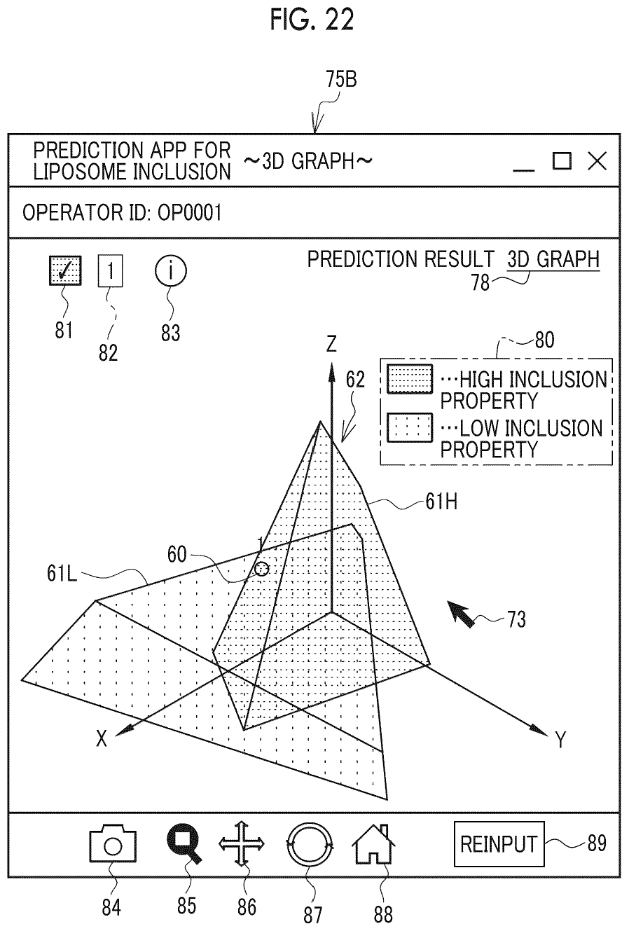

For example, as illustrated in FIG. 22, in a case where a predetermined operation of rotating a mouse wheel is performed after selecting the enlarging and reducing button 85 using the cursor 73, the feature value space for display 62 is enlarged or reduced. That is, the screen delivery controller 44 enlarges or reduces the feature value space for display 62 in accordance with the operation instruction of the operator OP. FIG. 22 illustrates a case where the feature value space for display 62 is enlarged from a default display state illustrated in FIG. 15 and the like.

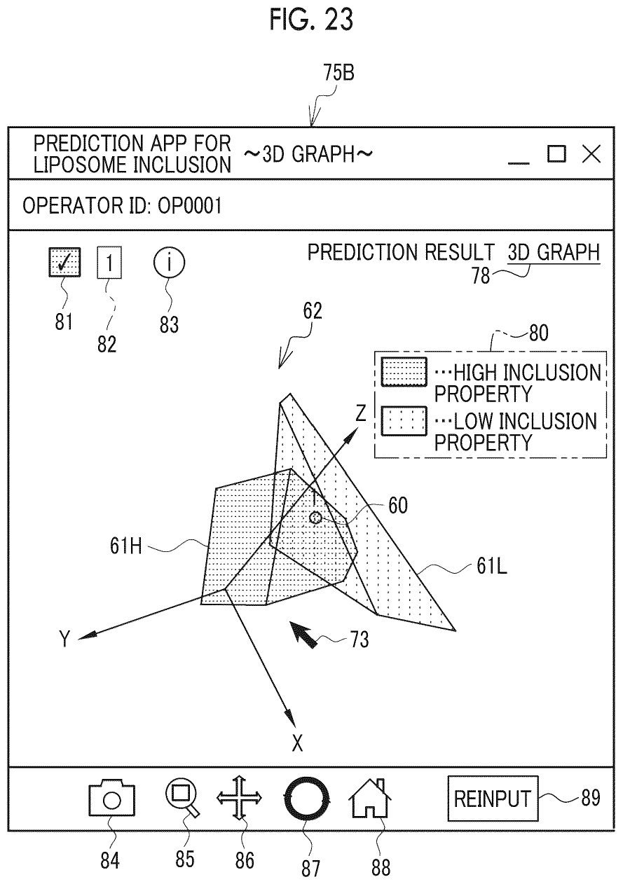

For example, as illustrated in FIG. 23, in a case where a predetermined operation of moving a mouse while right-clicking is performed after selecting the rotation button 87 using the cursor 73, the feature value space for display 62 is rotated. That is, the screen delivery controller 44 rotates the feature value space for display 62 in accordance with the operation instruction of the operator OP.

Functions of other buttons are as follows. First, the capture button 84 is a button for storing the feature value space for display 62 including the plot 60 and the regions 61H and 61L in the storage 25B in the form of, for example, a portable network graphics (PNG) file. The translation button 86 is a button for translating the feature value space for display 62 along any of upward, downward, leftward, or rightward directions. The home button 88 is a button for restoring the feature value space for display 62 to default display. A button for rotating the feature value space for display 62 with a Z axis fixed, and the like may be provided.

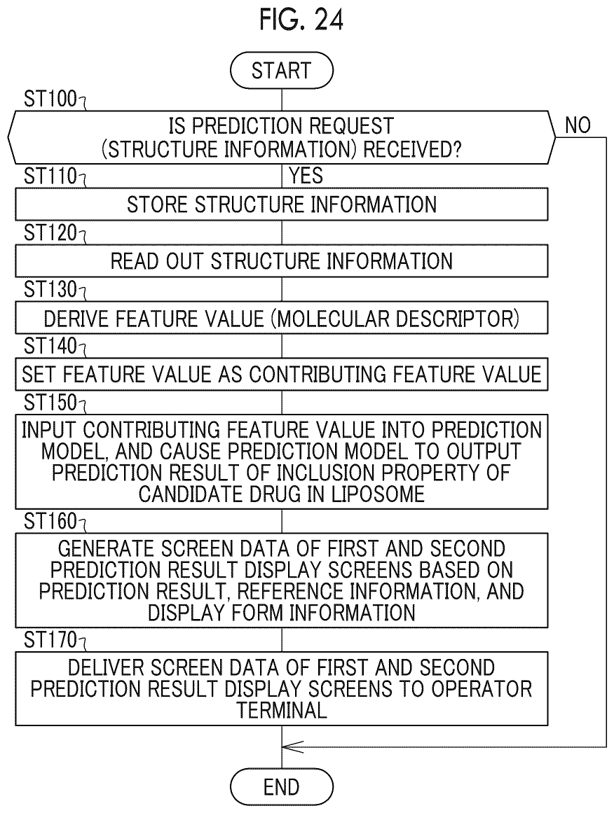

Next, an action of the above configuration will be described with reference to the flowchart illustrated in, for example, FIG. 24. In a case where the operation program 35 starts in the information processing apparatus 12, the CPU 27A functions as the request reception unit 40, the RW controller 41, the derivation unit 42, the prediction unit 43, and the screen delivery controller 44 as illustrated in FIG. 5. In a case where the prediction AP 65 starts in the operator terminal 13, the CPU 27B functions as the browser controller 67 as illustrated in FIG. 12.

The structure information input screen 70 illustrated in FIG. 13 is displayed on the display 29B of the operator terminal 13 under control of the browser controller 67. On the structure information input screen 70, the operator OP inputs desired structure information 16 in the input box 71 and selects the prediction button 72 using the cursor 73. Accordingly, the prediction request 15 is transmitted to the information processing apparatus 12 from the browser controller 67. As illustrated in FIG. 1, the prediction request 15 includes the structure information 16.

In the information processing apparatus 12, the structure information 16 included in the prediction request 15 is received by receiving the prediction request 15 via the request reception unit 40 (YES in step ST100). The structure information 16 included in the prediction request 15 is output to the RW controller 41 from the request reception unit 40 and is stored in the storage 25A under control of the RW controller 41 (step ST110). The terminal ID of the operator terminal 13 included in the prediction request 15 is output to the screen delivery controller 44 from the request reception unit 40.

The structure information 16 is read out from the storage 25A by the RW controller 41 (step ST120). The structure information 16 is output to the derivation unit 42 from the RW controller 41.

In the derivation unit 42, as illustrated in FIG. 6, the feature value 50 of the candidate drug 11C including the molecular descriptor is derived from the structure information 16 (step ST130). The feature value 50 is output to the prediction unit 43 from the derivation unit 42. The RW controller 41 reads out the prediction model 36 from the storage 25A and outputs the read prediction model 36 to the prediction unit 43.

In the prediction unit 43, as illustrated in FIG. 7, the molecular descriptor contributing to prediction of the inclusion property of the candidate drug 11C in the liposome 21 is selected among the plurality of types of molecular descriptors constituting the feature value 50, and the feature value 50 is set as the contributing feature value 50CO (step ST140). The contributing feature value 50CO is input into the prediction model 36. Accordingly, the prediction result 17 of the inclusion property of the candidate drug 11C in the liposome 21 is output from the prediction model 36 (step ST150). The prediction result 17 is output to the screen delivery controller 44 from the prediction unit 43. The RW controller 41 reads out the reference information 37 and the display form information 38 from the storage 25A and outputs the read reference information 37 and the read display form information 38 to the screen delivery controller 44.

In the screen delivery controller 44, as illustrated in FIG. 10, the dimensions of the contributing feature values 50CO of the candidate drug 11C and the reference drug 11R are reduced to obtain the three-dimensional feature value for display 50D. As illustrated in FIG. 11, the feature value space for display 62 is generated in accordance with the feature value for display 50D and the display form information 38. The feature value space for display 62 includes the plot 60 and the regions 61H and 61L. The plot 60 corresponds to the feature value for display 50D of the candidate drug 11C and has a display form corresponding to the prediction result 17. The regions 61H and 61L correspond to the plurality of feature values for display 50D of the plurality of reference drugs 11R and have the display form corresponding to the known inclusion properties of the reference drugs 11R in the liposome 21.

In the screen delivery controller 44, screen data of the first and second prediction result display screens 75A and 75B are generated (step ST160). The screen data of the first and second prediction result display screens 75A and 75B is delivered to the operator terminal 13 that is the transmitter of the prediction request 15, under control of the screen delivery controller 44 (step ST170).

As illustrated in FIGS. 14 and 15, in the operator terminal 13, the screen data of the first and second prediction result display screens 75A and 75B are reproduced under control of the browser controller 67, and the reproduced first and second prediction result display screens 75A and 75B are displayed on the display 29B. Accordingly, the prediction result 17 of the inclusion property of the candidate drug 11C in the liposome 21 and the known characteristic of the reference drug 11R are presented to the operator OP in a comparable manner.

The operator OP views the first and second prediction result display screens 75A and 75B. A determination as to whether or not to perform the practical task of the pharmaceutical preparation 20 using the candidate drug 11C is finally made by, for example, verifying validity of the prediction result 17 with reference to the feature value space for display 62.

As described above, the CPU 27A of the information processing apparatus 12 comprises the request reception unit 40, the derivation unit 42, the prediction unit 43, and the screen delivery controller 44. The request reception unit 40 receives the structure information 16 of the candidate drug 11C by receiving the prediction request 15. The derivation unit 42 derives the feature value 50 of the candidate drug 11C from the structure information 16. The prediction unit 43 predicts the inclusion property of the candidate drug 11C in the liposome 21 from the contributing feature value 50CO of the candidate drug 11C. The screen delivery controller 44 presents the prediction result 17 of the inclusion property of the candidate drug 11C in the liposome 21 and the known characteristic of the reference drug 11R in a comparable manner by delivering output of the screen data of the second prediction result display screen 75B to the operator terminal 13. Thus, the operator OP can easily compare the prediction result 17 with the known characteristic of the reference drug 11R. This can contribute to verification of the validity of the prediction result 17. Acceptance of the prediction result 17 can be increased. These effects are particularly useful for the operator OP who has little knowledge of the practical task of the liposome preparation and cannot empirically predict the characteristic of the drug 11 related to the liposome 21.

As illustrated in FIG. 15 and the like, the screen delivery controller 44 displays, in the feature value space for display 62, the plot 60 that corresponds to the feature value for display 50D of the candidate drug 11C and that has a display form corresponding to the prediction result 17. Thus, the operator OP can recognize a position of the candidate drug 11C in the feature value space for display 62 and the prediction result 17 of the inclusion property of the candidate drug 11C in the liposome 21 at a glance. This further helps verification of the validity of the prediction result 17.

As illustrated in FIG. 15 and the like, the screen delivery controller 44 displays, in the feature value space for display 62, the regions 61H and 61L that correspond to the plurality of feature values for display 50D of the plurality of reference drugs 11R and that have the display form corresponding to the known inclusion properties of the reference drugs 11R in the liposome 21. Thus, the operator OP can recognize the region 61H having a high inclusion property in the liposome 21 and the region 61L having a low inclusion property in the liposome 21 at a glance. This further helps verification of the validity of the prediction result 17.

As illustrated in FIGS. 16 and 18, the screen delivery controller 44, in accordance with reception of the structure information 16 of the new candidate drug 11C and derivation of the feature value 50 of the new candidate drug 11C, additionally displays the plot 60 corresponding to the feature value for display 50D of the new candidate drug 11C in the feature value space for display 62, in addition to the plot 60 corresponding to the feature value for display 50D of the candidate drug 11C of which the structure information 16 has been received so far. Thus, the operator OP can collectively view the prediction results 17 of the plurality of candidate drugs 11C. The operator OP can collectively verify the validity of the prediction results 17 of the plurality of candidate drug 11C. This further helps verification of the validity of the prediction result 17.

As illustrated in FIG. 18, the screen delivery controller 44 displays the plurality of plots 60 corresponding to the plurality of candidate drugs 11C such that the plurality of candidate drugs 11C can be identified from each other by the identification number 82. Thus, the operator OP can recognize which plot 60 corresponds to which candidate drug 11C without a mistake. This can reduce a possibility that the operator OP makes a mistake such as misidentifying the prediction result 17.

As illustrated in FIG. 19, the screen delivery controller 44 switches the plot 60 corresponding to the candidate drug 11C to be displayed or not displayed in accordance with the operation instruction of the operator OP. Thus, a demand of the operator OP for verifying the validity of the prediction result 17 for each candidate drug 11C can be fulfilled.

As illustrated in FIG. 11, the display form is a difference in color. Thus, visibility of the feature value space for display 62 from the operator OP can be increased. This further helps verification of the validity of the prediction result 17.

As illustrated in FIG. 20, the screen delivery controller 44 presents the coordinates 92 of the plot 60 corresponding to the feature value for display 50D of the candidate drug 11C in accordance with the operation instruction of the operator OP. Thus, the operator OP can accurately perceive the position of the candidate drug 11C in the feature value space for display 62.

As illustrated in FIG. 11 and the like, the feature value space for display 62 is a three-dimensional space. Thus, the validity of the prediction result 17 can be strictly verified compared to that in a case where the feature value space for display 62 is a one-dimensional space or a two-dimensional space.

As illustrated in FIGS. 22 and 23, the screen delivery controller 44 enlarges or reduces and/or rotates the feature value space for display 62 in accordance with the operation instruction of the operator OP. Thus, the operator OP can check a positional relationship between the plot 60 and the regions 61H and 61L in detail, which is difficult to understand with the default display. This further helps verification of the validity of the prediction result 17.

As illustrated in FIG. 21, the screen delivery controller 44 presents the chemical structural formula 95 of the candidate drug 11C in accordance with the operation instruction of the operator OP. Thus, the operator OP can verify the validity of the prediction result 17 while checking the chemical structural formula 95 of the candidate drug 11C.

As illustrated in FIG. 6, the feature value 50 is composed of the plurality of types of molecular descriptors. As illustrated in FIG. 7, the prediction unit 43 predicts the inclusion property of the candidate drug 11C in the liposome 21 from the contributing feature value 50CO obtained by selecting the molecular descriptor contributing to prediction of the inclusion property in the liposome 21 among the plurality of types of molecular descriptors. Thus, prediction accuracy of the inclusion property of the candidate drug 11C in the liposome 21 can be improved compared to that in a case where the inclusion property of the candidate drug 11C in the liposome 21 is predicted from the feature value 50 including all of the plurality of types of molecular descriptors.

As illustrated in FIG. 7, the prediction unit 43 inputs the contributing feature value 50CO of the candidate drug 11C into the prediction model 36 and causes the prediction model 36 to output the prediction result 17. Thus, the prediction result 17 of high prediction accuracy can be simply obtained.

As illustrated in FIG. 6, the feature value 50 includes the molecular descriptor as an clement. The molecular descriptor is generally widely used. Thus, the feature value 50 can be easily derived.

As illustrated in FIGS. 7 and 9, the characteristic is the inclusion property of the candidate drug 11C or the reference drug 11R in the liposome 21. Thus, an unnecessary cost can be reduced by preventing the operator OP from trying the practical task of the pharmaceutical preparation 20 using the candidate drug 11C having a relatively low inclusion property. This can be greatly beneficial to the operator OP in selecting the drug 11. This can also be greatly beneficial to the operator OP working in the pharmaceutical company in selecting the drug 11 in a case where the contract research organization is contracted to perform the practical task of the pharmaceutical preparation 20.

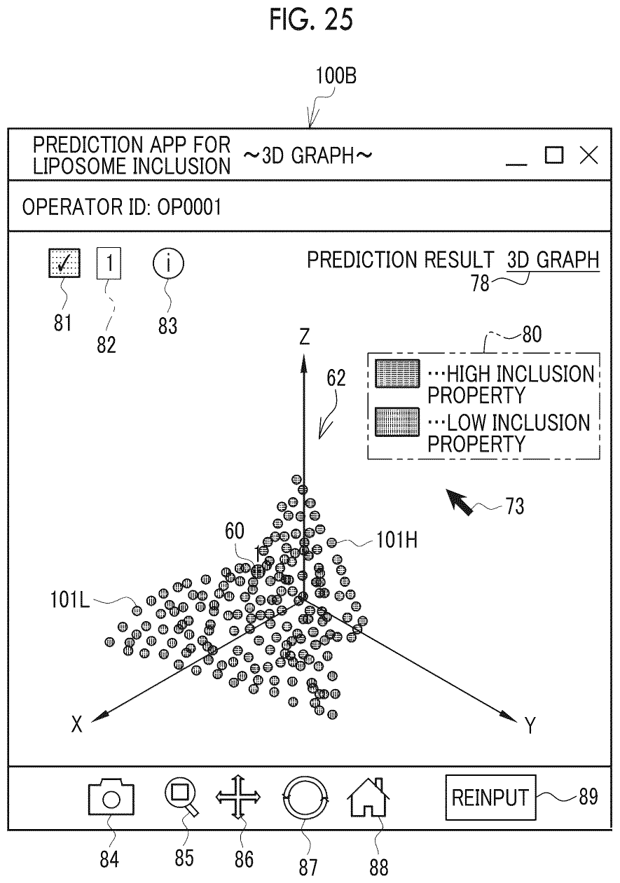

While the regions 61H and 61L corresponding to the plurality of feature values for display 50D of the plurality of reference drugs 11R are displayed in the feature value space for display 62, the disclosed technology is not limited to this. For example, as in the second prediction result display screen 100B illustrated in FIG. 25, a plurality of plots 101H and 101L corresponding to the plurality of feature values for display 50D of the plurality of reference drugs 11R may be displayed in the feature value space for display 62. The plot 101H corresponds to the feature value for display 50D of the reference drug 11R having a high inclusion property in the liposome 21. Meanwhile, the plot 101L corresponds to the feature value for display 50D of the reference drug 11R having a low inclusion property in the liposome 21. A display form of the plot 101H is green, and a display form of the plot 101L is orange, like the regions 61H and 61L of the first embodiment. In this case, the plot 60 has a larger size than the plots 101H and 101L.

The screen delivery controller 44 displays, in the feature value space for display 62, the plurality of plots 101H and 101L that correspond to the plurality of feature values for display 50D of the plurality of reference drug 11R and that have the display form corresponding to the known inclusion property in the liposome 21. This aspect can also contribute to verification of the validity of the prediction result 17.

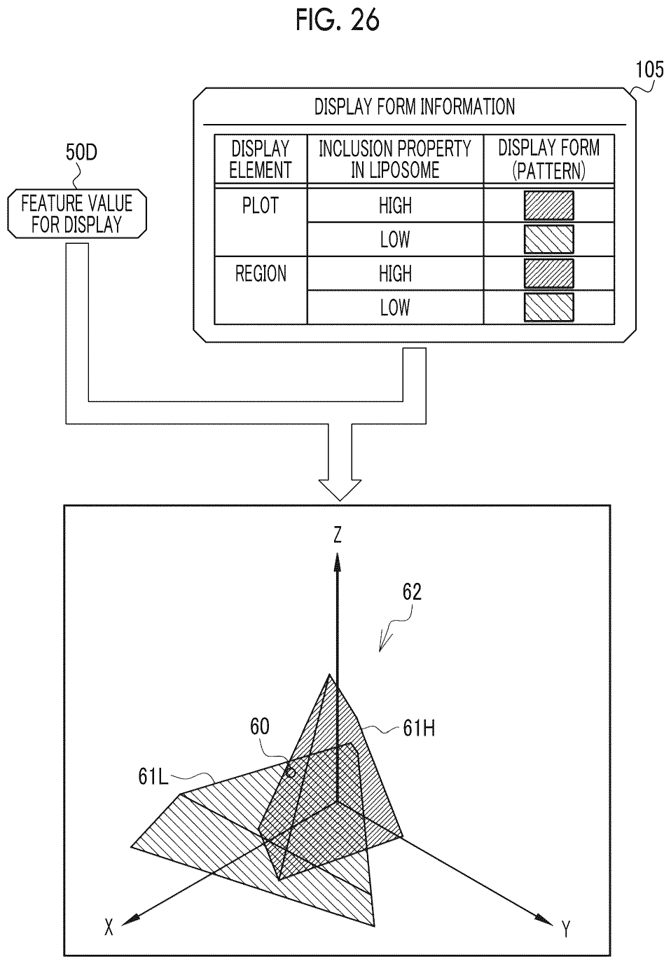

While a difference in color is illustrated as the display form, the disclosed technology is not limited to this. For example, as in display form information 105 illustrated in FIG. 26, the display form may be a difference in pattern. More specifically, the display form of the plot 60 corresponding to the feature value for display 50D of the candidate drug 11C is a pattern of diagonal straight lines arranged at equal intervals from the upper right to the lower left in a case where the inclusion property in the liposome 21 is high, and is a pattern of diagonal straight lines arranged at equal intervals from the upper left to the lower right in a case where the inclusion property in the liposome 21 is low. The display form of the region corresponding to the plurality of feature values for display 50D of the plurality of reference drugs 11R is a pattern (the region 61H) of diagonal straight lines arranged at equal intervals from the upper right to the lower left in a case where the inclusion property in the liposome 21 is high, and is a pattern (the region 61L) of diagonal straight lines arranged at equal intervals from the upper left to the lower right in a case where the inclusion property in the liposome 21 is low. This aspect can also increase the visibility of the feature value space for display 62 from the operator OP. While illustration is not provided, the display form may be a difference in color and pattern. The display form may be a difference in color density. Second Embodiment

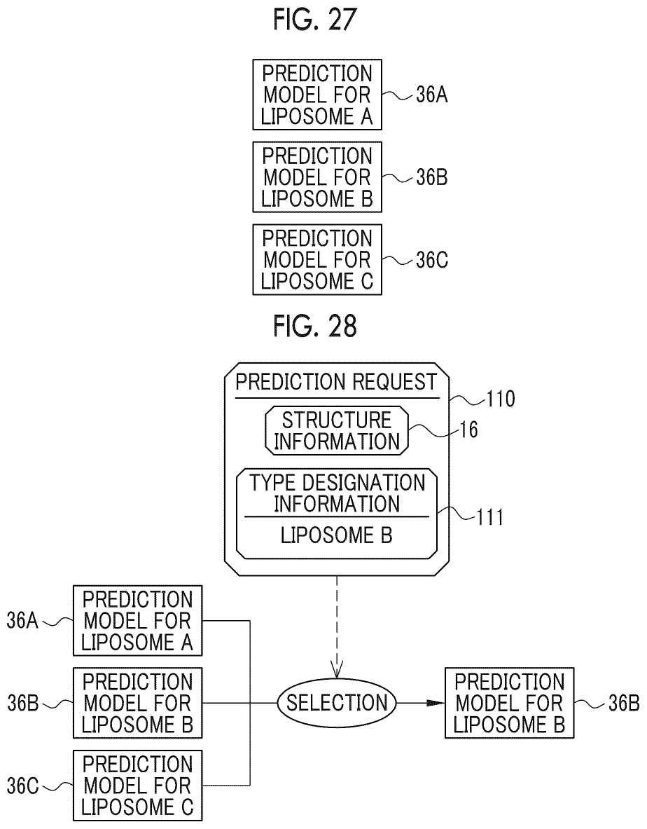

For example, as illustrated in FIG. 27, prediction models 36 corresponding to a plurality of types of liposomes 21 (liposomes A, B, and C), that is, a prediction model 36A for the liposome A, a prediction model 36B for the liposome B, and a prediction model 36C for the liposome C, are prepared in a second embodiment. For example, as illustrated in FIG. 28, a prediction request 110 of the second embodiment includes type designation information 111 in addition to the structure information 16. A type of the liposome 21 designated by the operator OP at the time of inputting the structure information 16 is registered in the type designation information 111. The prediction unit 43 selects and uses the prediction model 36 corresponding to the type of the liposome 21 of the type designation information 111. FIG. 28 illustrates a case where the designated type of the liposome 21 is the liposome B and the prediction model 36B for the liposome B is selected and used.

In the second embodiment, a plurality of types of the prediction models 36 are prepared in accordance with the types of the liposomes 21. The prediction unit 43 selects and uses the prediction model 36 corresponding to the type of the liposome 21. Thus, prediction corresponding to the type of the liposome 21 can be performed.

The chemical structural formula 95 may be displayed on the first prediction result display screen 75A together with the structure information 16 and the prediction result 17. Unnecessary structure information 16 and an unnecessary prediction result 17 may be configured to be removed on the first prediction result display screen 75A.

While the inclusion property, in the liposome 21, of the reference drug 11R registered in the reference information 37 is based on the prediction result 17 from the prediction model 36, the disclosed technology is not limited to this. The inclusion property in the liposome 21 obtained by actually conducting an experiment for the reference drug 11R may be registered.

While two levels of “high” and “low” of the inclusion property of the candidate drug 11C in the liposome 21 are illustrated as an output form of the prediction result 17, the disclosed technology is not limited to this. For example, the inclusion property of the candidate drug 11C in the liposome 21 may be shown with five levels of “very high”, “slightly high”, “medium”, “slightly low”, and “very low”. In this case, five regions are displayed in the feature value space for display 62.

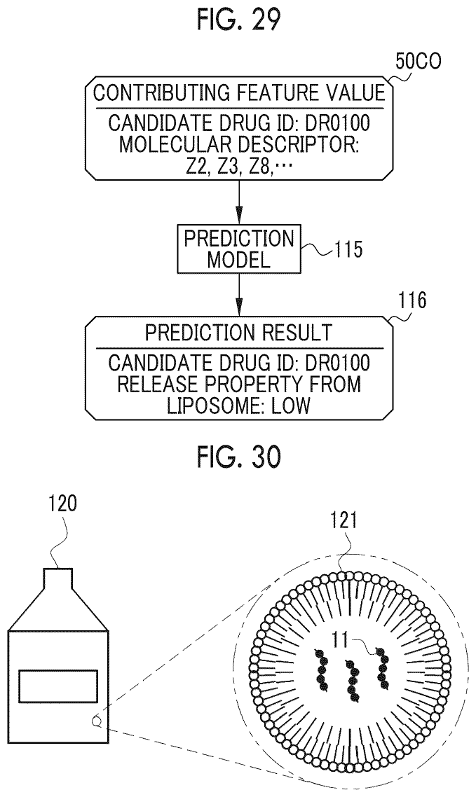

While the inclusion property in the liposome 21 is illustrated as the characteristic, the disclosed technology is not limited to this. For example, as in a prediction model 115 and a prediction result 116 illustrated in FIG. 29, a release property of the candidate drug 11C from the liposome 21 may be predicted as the characteristic. This aspect can also reduce an unnecessary cost by preventing the operator OP from trying the practical task of the pharmaceutical preparation 20 using the candidate drug 11C having a relatively high release property.

The characteristic may also be a size of an amount of inclusion of the candidate drug 11C in the liposome 21 or dynamics of the liposome 21 including the candidate drug 11C in a case where the pharmaceutical preparation 20 is applied to a living body (for example, whether or not the candidate drug 11C is easily absorbed in a liver). Alternatively, the characteristic may be safety in a case where the pharmaceutical preparation 20 is applied to a living body, or stability of the candidate drug 11C included in the liposome 21 (for example, whether or not a time from inclusion in the liposome 21 to release is short). A plurality of types of characteristics may be collectively predicted. In this case, the feature value space for display 62 is also generated for each of the plurality of types of characteristics.

Input of the chemical structural formula 95 may be received as the structure information 16 instead of the illustrated SMILES string. Input of a molecular design limited (MOL) file or a structure data format (SDF) file may also be received.

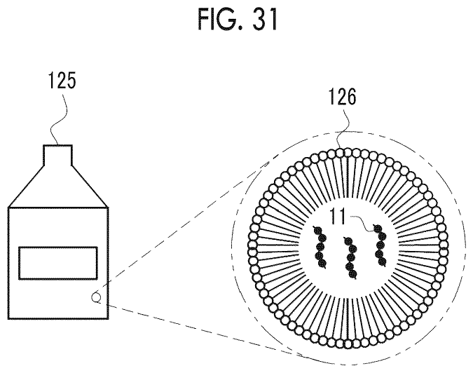

While the liposome 21 is illustrated as the vesicle, the disclosed technology is not limited to this. A lipid nanoparticle 121 may be used as in a pharmaceutical preparation 120 illustrated in, for example, FIG. 30. The lipid nanoparticle 121 is literally a particle that is composed of a lipid and that has a nanoscale diameter. A micelle 126 may also be used as in a pharmaceutical preparation 125 illustrated in, for example, FIG. 31. The micelle 126 is a particle that is composed of a surfactant and that has a nanoscale diameter.

The candidate substance and a reference substance are not limited to the illustrated candidate drug 11C and the illustrated reference drug 11R. For example, an active ingredient of a cosmetic product or an active ingredient of a nutritional supplement (a supplement) may be used.

The feature value 50 may include, in addition to or instead of the illustrated molecular descriptors, a molecular weight, the number of carbon-carbon bonds, the number of carbon-oxygen bonds, the number of carbon-nitrogen bonds, the number of six-membered rings, and the like. The feature value 50 may include various parameters obtained by experiment and/or simulation, such as solubility, an acid dissociation constant, and the number of charges. The feature value space for display 62 may be a one-dimensional space or a two-dimensional space.

The information processing apparatus 12 may be installed in the pharmaceutical facility or may be installed in a data center independent of the pharmaceutical facility.

Data that is a basis of the first and second prediction result display screens 75A and 75B, such as the prediction result 17, the reference information 37, and the display form information 38, may be delivered to the operator terminal 13 instead of delivering the screen data of the first and second prediction result display screens 75A and 75B to the operator terminal 13. In this case, in the operator terminal 13, the first and second prediction result display screens 75A and 75B are generated based on the prediction result 17, the reference information 37, and the display form information 38 under control of the browser controller 67, and the first and second prediction result display screens 75A and 75B are displayed on the display 29B.

A method of presenting the prediction result 17 of the characteristic of the candidate drug 11C and the known characteristic of the reference drug 11R to the operator OP is not limited to the illustrated presentation based on delivery of the screen data. The prediction result 17 of the characteristic of the candidate drug 11C and the known characteristic of the reference drug 11R, that is, the feature value space for display 62 including the plot 60 and the regions 61H and 61L, may be presented to the operator OP in a printed manner on a paper medium, or the feature value space for display 62 including the plot 60 and the regions 61H and 61L may be presented to the operator OP by attaching the feature value space for display 62 to an electronic mail and transmitting the electronic mail to the operator terminal 13.

A hardware configuration of the computer constituting the information processing apparatus 12 according to the disclosed technology can be variously modified. For example, the information processing apparatus 12 may be composed of a plurality of separated computers as hardware for the purpose of improving processing ability and reliability. For example, functions of the request reception unit 40 and the RW controller 41 and functions of the derivation unit 42, the prediction unit 43, and the screen delivery controller 44 are provided in a distributed manner between two computers. In this case, the information processing apparatus 12 is composed of two computers. The operator terminal 13 may have a part or all of the functions of the information processing apparatus 12.

The hardware configuration of the computer of the information processing apparatus 12 can be appropriately changed in accordance with required performance such as processing ability, safety, and reliability. Not only the hardware but also the APs such as the operation program 35 may be duplicated or stored in a distributed manner between a plurality of storages for the purpose of securing safety and reliability.

In each of the embodiments, for example, a hardware structure of a processing unit that executes various types of processing, such as the request reception unit 40, the RW controller 41, the derivation unit 42, the prediction unit 43, the screen delivery controller 44, and the browser controller 67, can use the following various processors. The various processors include, in addition to the CPUs 27A and 27B that are general-purpose processors functioning as various processing units by executing software (the operation program 35 and the prediction AP 65) as described above, a programmable logic device (PLD) such as a field programmable gate array (FPGA) that is a processor having a circuit configuration changeable after manufacture, a dedicated electric circuit such as an application specific integrated circuit (ASIC) that is a processor having a circuit configuration dedicatedly designed to execute specific processing, and the like.

One processing unit may be composed of one of the various processors or may be composed of a combination of two or more processors of the same type or different types (for example, a combination of a plurality of FPGAs and/or a combination of a CPU and an FPGA). A plurality of processing units may be composed of one processor.

A first example of the plurality of processing units composed of one processor is, as represented by a computer such as a client and a server, a form in which one processor is composed of a combination of one or more CPUs and software and the processor functions as the plurality of processing units. A second example is, as represented by a system on chip (SoC) or the like, a form of using a processor that implements functions of the entire system including the plurality of processing units in one integrated circuit (IC) chip. Accordingly, various processing units are composed of one or more of the various processors as the hardware structure.

More specifically, the hardware structure of the various processors can use an electric circuit (circuitry) obtained by combining circuit elements such as semiconductor elements.

From the above description, the technology according to the following appendices can be perceived.

### Appendix 1

An information processing apparatus comprising:
a processor,
in which the processor is configured to:
   receive structure information of a candidate substance to be included in a vesicle;
   derive a feature value of the candidate substance from the structure information;
   predict a characteristic of the candidate substance from the feature value of the candidate substance; and
   present a prediction result of the characteristic of the candidate substance and a known characteristic of a reference substance in a comparable manner.

### Appendix 2

The information processing apparatus according to Appendix 1, in which the processor is configured to display, in a feature value space, a plot that corresponds to the feature value of the candidate substance and that has a display form corresponding to the prediction result.

### Appendix 3

The information processing apparatus according to Appendix 2, in which the processor is configured to display, in the feature value space, a region that corresponds to a plurality of feature values of a plurality of the reference substances and that has a display form corresponding to the known characteristic.

### Appendix 4

The information processing apparatus according to Appendix 2, in which the processor is configured to display, in the feature value space, a plurality of plots that correspond to a plurality of feature values of a plurality of the reference substances and that have a display form corresponding to the known characteristic.

### Appendix 5

The information processing apparatus according to any one of Appendices 2 to 4, in which the processor is configured to, in accordance with reception of the structure information of a new candidate substance and derivation of a feature value of the new candidate substance, additionally display a plot corresponding to the feature value of the new candidate substance in the feature value space, in addition to the plot corresponding to the feature value of the candidate substance of which the structure information has been received so far.

### Appendix 6

The information processing apparatus according to Appendix 5, in which the processor is configured to display a plurality of plots corresponding to a plurality of the candidate substances such that the plurality of candidate substances are identifiable from each other.

### Appendix 7

The information processing apparatus according to any one of Appendices 2 to 6, in which the processor is configured to switch the plot corresponding to the candidate substance to be displayed or not displayed in accordance with an operation instruction of an operator.

### Appendix 8

The information processing apparatus according to any one of Appendices 2 to 7, in which the display form is a difference in color and/or pattern.

### Appendix 9

The information processing apparatus according to any one of Appendices 2 to 8, in which the processor is configured to present coordinates of the plot corresponding to the feature value of the candidate substance in accordance with an operation instruction of an operator.

### Appendix 10

The information processing apparatus according to any one of Appendices 2 to 9, in which the feature value space is a three-dimensional space.

### Appendix 11

The information processing apparatus according to Appendix 10, in which the processor is configured to enlarge or reduce and/or rotate the feature value space in accordance with an operation instruction of an operator.

### Appendix 12

The information processing apparatus according to any one of Appendices 1 to 11, in which the processor is configured to present a chemical structural formula of the candidate substance in accordance with an operation instruction of an operator.

### Appendix 13

The information processing apparatus according to any one of Appendices 1 to 12,
in which the feature value is composed of a plurality of types of elements, and
the processor is configured to predict the characteristic of the candidate substance from a contributing feature value obtained by selecting an element contributing to prediction of the characteristic from the plurality of types of elements.

### Appendix 14

The information processing apparatus according to any one of Appendices 1 to 13, in which the processor is configured to input the feature value of the candidate substance into a machine learning model and cause the machine learning model to output the prediction result.

### Appendix 15

The information processing apparatus according to Appendix 14,
in which a plurality of types of the machine learning model are prepared in accordance with a type of the vesicle, and
the processor is configured to select and use the machine learning model corresponding to the type of the vesicle.

### Appendix 16

The information processing apparatus according to any one of Appendices 1 to 15, in which the feature value includes a molecular descriptor as an element.

### Appendix 17

The information processing apparatus according to any one of Appendices 1 to 16, in which the characteristic includes at least any one of an inclusion property of the candidate substance or the reference substance in the vesicle or a release property of the candidate substance or the reference substance from the vesicle.

### Appendix 18

The information processing apparatus according to any one of Appendices 1 to 17, in which the vesicle is any of a liposome, a lipid nanoparticle, or a micelle.

In the disclosed technology, the above various embodiments and/or various modification examples can be appropriately combined with each other. Not only the embodiments but also various configurations not departing from the gist of the disclosed technology may be employed. The disclosed technology also applies to, in addition to the program, a storage medium storing the program in a non-transitory manner.

The described contents and the illustrated contents shown above are detailed descriptions of parts according to the disclosed technology and are merely an example of the disclosed technology. For example, description related to the above configurations, functions, actions, and effects is description related to an example of configurations, functions, actions, and effects of the parts according to the disclosed technology. Thus, unnecessary parts may be removed, new elements may be added, or parts may be replaced in the described contents and the illustrated contents shown above without departing from the gist of the disclosed technology. Description related to common technical knowledge or the like that does not require particular description in terms of embodying the disclosed technology is omitted in the described contents and the illustrated contents shown above, in order to avoid complication and facilitate understanding of the parts according to the disclosed technology.

In the present specification, “A and/or B” is synonymous with “at least one of A or B”. That is, “A and/or B” may mean only A, only B, or a combination of A and B. In the present specification, the same approach as “A and/or B” applies to an expression of three or more matters connected with “and/or”.

All documents, patent applications, and technical standards described in the present specification are incorporated in the present specification by reference to the same extent as in a case where individual documents, patent applications, and technical standards are specifically and individually indicated to be incorporated by reference.

What is claimed is:

1. An information processing apparatus comprising:
a processor,
wherein the processor is configured to:
receive structure information of a candidate substance to be included in a vesicle;
derive a feature value of the candidate substance from the structure information;
predict a characteristic of the candidate substance from the feature value of the candidate substance; and
present a prediction result of the characteristic of the candidate substance and a known characteristic of a reference substance in a comparable manner.

2. The information processing apparatus according to claim 1,
wherein the processor is configured to display, in a feature value space, a plot that corresponds to the feature value of the candidate substance and that has a display form corresponding to the prediction result.

3. The information processing apparatus according to claim 2,
wherein the processor is configured to display, in the feature value space, a region that corresponds to a plurality of feature values of a plurality of the reference substances and that has a display form corresponding to the known characteristic.

4. The information processing apparatus according to claim 2,
wherein the processor is configured to display, in the feature value space, a plurality of plots that correspond to a plurality of feature values of a plurality of the reference substances and that have a display form corresponding to the known characteristic.

5. The information processing apparatus according to claim 2,
wherein the processor is configured to, in accordance with reception of the structure information of a new candidate substance and derivation of a feature value of the new candidate substance, additionally display a plot corresponding to the feature value of the new candidate substance in the feature value space, in addition to the plot corresponding to the feature value of the candidate substance of which the structure information has been received so far.

6. The information processing apparatus according to claim 5,
wherein the processor is configured to display a plurality of plots corresponding to a plurality of the candidate substances such that the plurality of candidate substances are identifiable from each other.

7. The information processing apparatus according to claim 2,
wherein the processor is configured to switch the plot corresponding to the candidate substance to be displayed or not displayed in accordance with an operation instruction of an operator.

8. The information processing apparatus according to claim 2,
wherein the display form is a difference in color and/or pattern.

9. The information processing apparatus according to claim 2,
wherein the processor is configured to present coordinates of the plot corresponding to the feature value of the candidate substance in accordance with an operation instruction of an operator.

10. The information processing apparatus according to claim 2,
wherein the feature value space is a three-dimensional space.

11. The information processing apparatus according to claim 10,
wherein the processor is configured to enlarge or reduce and/or rotate the feature value space in accordance with an operation instruction of an operator.

12. The information processing apparatus according to claim 1,
wherein the processor is configured to present a chemical structural formula of the candidate substance in accordance with an operation instruction of an operator.

13. The information processing apparatus according to claim 1,
wherein the feature value is composed of a plurality of types of elements, and
the processor is configured to predict the characteristic of the candidate substance from a contributing feature value obtained by selecting an element contributing to prediction of the characteristic from the plurality of types of elements.

14. The information processing apparatus according to claim 1,
wherein the processor is configured to input the feature value of the candidate substance into a machine learning model and cause the machine learning model to output the prediction result.

15. The information processing apparatus according to claim 14,
wherein a plurality of types of the machine learning model are prepared in accordance with a type of the vesicle, and
the processor is configured to select and use the machine learning model corresponding to the type of the vesicle.

16. The information processing apparatus according to claim 1,
wherein the feature value includes a molecular descriptor as an element.

17. The information processing apparatus according to claim 1,
wherein the characteristic includes at least any one of an inclusion property of the candidate substance or the reference substance in the vesicle or a release property of the candidate substance or the reference substance from the vesicle.

18. The information processing apparatus according to claim 1,
wherein the vesicle is any of a liposome, a lipid nanoparticle, or a micelle.

19. An operation method of an information processing apparatus, the method comprising:
receiving structure information of a candidate substance to be included in a vesicle;
deriving a feature value of the candidate substance from the structure information;
predicting a characteristic of the candidate substance from the feature value of the candidate substance; and
presenting a prediction result of the characteristic of the candidate substance and a known characteristic of a reference substance in a comparable manner.

20. A non-transitory computer-readable storage medium storing an operation program of an information processing apparatus, the program causing a computer to execute a process comprising:
receiving structure information of a candidate substance to be included in a vesicle;
deriving a feature value of the candidate substance from the structure information;
predicting a characteristic of the candidate substance from the feature value of the candidate substance; and
presenting a prediction result of the characteristic of the candidate substance and a known characteristic of a reference substance in a comparable manner.
